(19) United States
(12) Patent Application Publication
(10) Pub. No.: US 2010/0203567 A1
(43) Pub. Date: Aug. 12, 2010

(54) ESBL Detection Kit and Method

(76) Inventors: Natalie Michelle Mitchell, Attikis (GR); Guduru Gopal Rao, London (GB); James Wong, London (GB)

Correspondence Address: SMITH, GAMBRELL & RUSSELL, 1130 CONNECTICUT AVENUE, N.W., SUITE 1130, WASHINGTON, DC, 20036, US

(21) Appl. No.: 12/531,321

(22) PCT Filed: Mar. 17, 2008
(86) PCT No.: PCT/GB08/00945

§ 371 Date: Mar. 31, 2010

(30) Foreign Application Priority Data
Mar. 16, 2007 (GB) 0705146.9

(51) Int. Cl. C12Q 1/26

(52) U.S. Cl. 435/25

(57) ABSTRACT

The invention provides methods and detection kits for detecting extended spectrum β-lactamase producers (ESBLs). The kits typically comprise a carrier containing: a)at least one sugar; b) at least one antibiotic for killing Gram positive bacteria; c) at least one antifungal compound; d) a mixture of cefpodoxime and at least one additional antibiotic for killing Gram negative bacteria, and/or a mixture of cefotaxime and ceftazidime; and e) a pH indicator. The methods and kits can be used to by non-specialist health professionals to give rapid results at relatively low cost.

### TECHNICAL FIELD

This invention relates to the detection of bacteria which are extended spectrum β-lactamase producers (ESBLs). In particular, this invention relates to a detection kit and a method, for detecting ESBLs.

### BACKGROUND ART

ESBLs are bacteria which produce extended spectrum β-lactamase enzymes which are capable of hydrolysing the β-lactam group of commonly used β-lactam antibiotics. This confers the bacteria with a resistance to a wide range of commonly used antibiotics such as penicillins, monobactams and 2^{nd}/3^{′d} generation cephalosporins.

ESBLs are becoming increasingly prevalent in both the community and in hospitals around the world including the UK. Major outbreaks have recently been reported in UK hospitals including an outbreak which was associated with 19% mortality. Treatment options for patients in such outbreaks are limited as a result of the ESBLs' resistance to common β-lactam antibiotics making such outbreaks difficult and expensive to control.

The current trends suggest that the prevalence of ESBLs is likely to increase which will lead to higher treatment costs and extended hospital stays for a greater number of patients. Accordingly, there is a need for measures to prevent/limit future outbreaks of infection by ESBLs.

As with any transmissible infection, the key to preventing/controlling the spread of infection by ESBLs is to have an effective detection method to identify infected individuals or individuals carrying the bacteria in their gastrointestinal tract so as to limit transmission from the infected individuals/carriers by implementing infection control measures.

Additionally, It has been suggested that the food supply might be a reservoir of ESBL-producers that infect humans. (Carattoli A. et al. 2008, Perez et al. 2007). Diverse ESBL-producing bacteria, including those containing CTX-M-15, have been identified in the following farm animals: pigs, cattle, rabbits, pigeons, poultry [Duan et al. 2006, Mesa et al.2006. Meunier et al. 2006]. ESBL-producing bacteria have not been identified in vegetables yet. However, a cross-contamination of vegetables by animal products in a farm-environment appears possible.

A number of detection methods for detecting ESBLs are known. For example, it is know to detect ESBLs by detecting the presence of DNA sequences that are indicative for these bacteria e.g. using microarray profiles or detecting genetic sequences of ESBL genes. Other methods rely on the fact that β-lactamases produced by ESBLs can cleave certain coloured compounds e.g. certain cephem compounds which leads to a detectable change in their absorption spectrum. Yet further methods detect ESBLs by their resistance to a specific set of antibiotics for example using the Double-disk diffusion test or a MacConkey agar containing ceftazidime.

There are several problems with the known detection methods. Firstly, significant expertise is required to carry out the tests; typically, the known tests require the involvement of a clinical microbiologist with access to a laboratory infrastructure. This makes using the methods expensive and thus it is standard practice to only test pre-screened individuals rather than, for example, mass-screening of patients upon admission to hospital. Apart from the (very expensive) DNA testing methods, all of the known methods require pure cultures of bacteria which have to be obtained from the individual and then grown over at least 48 hours prior to testing. This means that these known tests cannot be used to obtain rapid results which would allow infection control measures to be implemented at the earliest possible opportunity.

### DISCLOSURE OF THE INVENTION

The present invention aims to ameliorate at least some of the above problems and preferably to provide a detection kit and method which can easily be used by a non-specialist health professional e.g. a nurse to give rapid results at a lower cost than the known methods.

Accordingly, in a first aspect, the present invention provides a detection kit for detecting the presence of ESBLs, the kit comprising a carrier containing:

a) at least one sugar;

b) at least one antibiotic for killing Gram positive bacteria;

c) at least one antifungal compound;

d) a mixture of cefpodoxime and at least one additional antibiotic for killing Gram negative bacteria, and/or a mixture of cefotaxime and ceftazidime; and

e) a pH indicator.

This kit can be used to selectively detect the presence of ESBLs in a clinical specimen introduced to the carrier. The antibiotics/antifungal present in the carrier will kill all non-resistant bacteria/fungi i.e. those which are not ESBLs. ESBLs present in the clinical specimen will metabolise the sugar to generate hydrogen ions which will decrease the pH of the carrier thus causing a change in colour of the pH indicator. Accordingly, the detection kit of the first aspect of the present invention provides a clear indication that ESBLs are present in the specimen by a colour change which can be visually observed by any healthcare professional. This detection kit will give a relatively rapid result; the inoculated carrier can be incubated e.g. for 18-24 hours at the end of which there will be an indication (i.e. colour change) if any ESBLs are present.

The carrier may be an agar medium but preferably the carrier is a liquid medium. More preferably the carrier is water (e.g. distilled/sterile water) and/or a sodium chloride solution (saline) and most preferably, the sugar, antibiotics/antifungal(s) and pH indicator are dissolved in the carrier to form an aqueous solution. Using a liquid carrier provides a number of advantages.

Firstly, an agar plate carrying antibiotics requires refrigeration prior to use to prevent the agar from drying out (which can lead to deterioration of the antibiotics). In contrast, using a liquid carrier improves stability of the antibiotics and the kit does not require refrigeration prior to use.

Secondly, the specimen can easily be introduced into the liquid carrier e.g. by stirring a swab carrying the clinical specimen in the liquid carrier. This can be carried out by any health professional e.g. at a patient's bedside with minimal contamination risk. In contrast, introducing a specimen to an agar plate carrier is typically undertaken by trained, skilled staff in a laboratory using stringent aseptic techniques. The ease of use of the kit (and its consequential reduced cost) will allow mass screening of patients rather than screening of only pre-selected individuals. This will facilitate infection control.

Furthermore, in agar techniques, the inoculated plate requires immediate incubation whereas immediate incubation is not critical for a liquid carrier.

The carrier may also include a buffer such as a sodium phosphate buffer. The buffer helps to resist small changes in the pH of the carrier thus reducing the incidence of a false positive result.

The at least one sugar preferably includes D-glucose (dextrose). This is because all ESBLs can metabolise D-glucose and therefore the detection kit can be used to detect the presence of any resistant ESBL. Alternatively, the at least one sugar may include another sugar such as lactose. This would allow the kit to selectively test for lactose-fermenting ESBL.

In preferred embodiments, the carrier further includes a growth enhancer for assisting growth of the ESBLs during incubation of the detection kit. Suitable growth enhancers include peptone powder, nutrient broth powder and yeast extract.

The antibiotic(s) for killing Gram positive bacteria is/are selected such that any/all Gram positive bacteria introduced into the carrier from a specimen are killed i.e. at least one broad-spectrum antibiotic for killing Gram positive bacteria is used. The at least one antibiotic for killing Gram positive bacteria may be, for example, a glycopeptide antibiotic such as vancomycin or teicoplanin.

The antifungal/antimycotic compound(s) is/are selected such that any/all fungi introduced into the carrier from a specimen are killed i.e. at least one broad-spectrum antifungal compound is used. The at least one antifungal/antimycotic may be, for example, a polyene antimycotic such as amphotericin or nystatin and/or an imidazole/triazole antimycotic such as fluconazole.

The cefpodoxime/at least one additional antibiotic for killing Gram negative bacteria mixture and/or the cefotaxime/ceftazidime mixture are used in the detection kit to kill all Gram negative bacteria (except ESBLs). Cefpodoxime and/or cefotaxime/ceftazidime are used because all ESBLs are resistant to cefpodoxime and cefotaxime/ceftazidime in in vitro tests. ESBLs can show variable resistance to other cephalosporin antibiotics in in vitro tests (although they will be resistant in vivo) and this variable resistance could lead to a false negative result if other cephalosporin antibiotics are used. By using cefpodoxime and/or a cefotaxime/ceftazidime mixture, the detection kit of the present invention ensures that a false negative result is not obtained.

When cefpodoxime is used, at least one additional antibiotic for killing Gram negative bacteria is used to kill those Gram negative bacteria which are not killed by cefpodoxime e.g. Pseudomonas aeruginosa. This additional antibiotic is not necessary when a mixture of cefotaxime/ceftazidime is used because this mixture is effective against a wider range of Gram negative bacteria than cefpodoxime alone. However, it is preferable to use at least one additional antibiotic for killing Gram negative bacteria with the cefotaxime/ceftazidime mixture.

The antibiotic(s) for killing Gram negative bacteria is/are selected such any/all Gram negative bacteria (or at least those not killed by cefpodoxime or cefotaxime/ceftazidime) introduced into the carrier from a clinical specimen are killed i.e. a broad spectrum antibiotic for killing Gram negative bacteria is used. The at least one antibiotic for killing Gram negative bacteria may be, for example, a monobactam such as aztreonam (AZT).

This cocktail of antibiotics/antifungal(s) in the carrier is selected to kill all bacteria/fungi introduced into the carrier in the specimen except ESBLs leaving the ESBLs to metabolise the sugar resulting in the pH decrease and indicator colour change. The inclusion of the antibiotics/antifungal(s) in the carrier reduces/prevents the incidence of a false positive result from the kit (caused by the metabolism of bacteria/fungi other than ESBLs.)

A preferred combination of antibiotics/antifungal(s) in the carrier is the combination of vancomycin/teicoplanin, amphotericin, and cefpodoxime/AZT or cefotaxime/ ceftazidime (optionally with AZT). Most preferred is vancomycin, amphotericin, cefpodoxime and AZT. This combination provides a kit which is highly selective for ESBLs and prevents a false positive or false negative result.

Each antibiotic/antifungal is preferably included in the carrier in an amount corresponding to its minimum inhibitory concentration (although greater amounts can be used). For example, it is preferable to include vancomycin in the carrier at a concentration of greater than 4 mg/l, amphotericin at a concentration of greater than 2 mg/l, AZT at a concentration of greater than 4 mg/l and cefpodoxime at a concentration of greater than 1 mg/l.

The pH indicator can be any indicator which shows a colour change as the pH changes from substantially neutral to acidic. Suitable indicators include bromothymol blue which has a colour transition pH between 6.0 and 7.6, phenol red which has a colour transition pH between 6.8 and 8.4 and neutral red which has a colour transition pH between 6.8 and 8.0. Phenol red is the preferred pH indicator because it exhibits a distinct colour change from red to yellow as the pH changes from neutral to acidic.

Preferably, the carrier is contained within a vessel such as a bottle. More preferably, the vessel is transparent. This allows for easy visual inspection of the contents of the vessel to detect a colour change indicating a change in pH. Most preferably, the vessel is a sealable by a lid e.g. a screw-top or press-fit lid. This prevents contamination of the carrier both before and after inoculation with the clinical specimen.

In a second aspect the present invention provides a method for detecting the presence of ESBLs comprising:

a) inoculating the carrier of a detection kit according to the first aspect,

b) incubating the detection kit;

c) detecting a colour change in the pH indicator in the carrier.

The kits and methods described herein may be applied to any sample in which it is desired to test for the presence of ESBLs.

As well as clinical samples, it will be understood than the invention may likewise be applied to the testing of farm animals, animal products and vegetables. Taking samples from animals, animal products or vegetables to inoculate growth media is a standard procedure in animal/food microbiology and in the light of the present disclosure those skilled in the art will be readily able to adapt such procedures to inoculate the ESBL broth of the invention. Samples of food could be added directly or after a dilution step to the ESBL broth before incubation. Alternatively, swabs could be used to take samples from the body surface of animals.

Thus in one embodiment the sample is a clinical specimen such as a body fluid.

In other embodiments the sample may be an environmental sample (e.g. from a hospital or farm) or a foodstuff such as an animal product (e.g. from eggs, milk, meat, milk-products, processed mea) or a vegetable (e.g. lettuce)

In another embodiment the sample may be a test sample derived from an animal, especially a commercial or farm animal e.g. livestock, cattle (cows and oxen), pigs, rabbits, poultry (broiler chickens, egg-laying hens, turkeys, geese, ducks, other fowl, pheasants, partridges, emus), pigeons, llamas, ostriches, horses, bison, buffalo, sheep, goats, deer, mink etc.

Preferably, the carrier of the detection kit is a liquid carrier and the inoculating is carried out by introducing the sample (e.g. clinical specimen) into the liquid carrier e.g. by dipping/stirring a swab carrying the specimen in the carrier.

Preferably, the incubating is carried out for 18-24 hours at 36-37° C.

Preferably, the detecting step is carried out by visually observing the colour of the pH indicator in the carrier. For example, using phenol red, a colour change from red to yellow would be observed if any ESBL is present. If no colour change is observed, it can be assumed that no ESBL was present in the sample (e.g. clinical specimen).

The invention will now be further described with reference to, the following non-limiting Figures and Examples. Other embodiments of the invention will occur to those skilled in the art in the light of these.

The disclosure of all references cited herein, inasmuch as it may be used by those skilled in the art to carry out the invention, is hereby specifically incorporated herein by cross-reference.

### EXAMPLES

### Example 1

### A Batch of “Broth” for the Detection Kit was Made Up by Mixing

250 ml of sodium phosphate buffered saline;

750 ml of distilled water;

4 mg/l vancomycin;

2 mg/l amphotericin;

4 mg/l AZT;

1 mg/l cefpodoxime sodium;

1.4 ml of (2%) phenol red pH indicator; and

15 g of (1%) peptone powder.

5 ml of the broth was added to a universal sample bottle (transparent plastic bottle with screw cap) to yield a detection kit.

### Example 2

In order to test the ability of the detection kit to selectively detect ESBLs, 50 μl of 1×10⁸ cfu/ml (0.5 McFarland) dilutions of national cultured type strains (NCTC) of various organisms was used to inoculate the carrier of a respective detection kit. (This dilution of organisms was selected as it is the minimum concentration considered to be a significant growth in urine samples.) The inoculated detection kits were incubated for 18-24 hours at 36-37° C. The kits were inspected to detect a colour change in the pH indicator.

The results are shown in Table 1.

TABLE 1

| Organism | NCTC number | Colour change |
|---|---|---|
| E. coli | 10536 | No |
| Enterococcus faecalis | 12697 | No |
| Staphylococcus aureus | 8532 | No |
| MRSA 15 | 13142 | No |
| Proteus mirabilis | 11938 | No |
| Pseudomonas aeroginosa | 10662 | No |
| E. coli (ESBL) | 13351 | Yes |
| E. coli (ESBL) | 13352 | Yes |
| E. coli (ESBL) | 13353 | Yes |

This test shows that the detection kit was selective and only gave a positive result (indicated by a colour change) when the carrier was inoculated with ESBL.

### Example 3

In order to test the ability of the kit to detect small numbers of organisms, 10 μl of an overnight nutrient broth culture of ESBL was added to the liquid carrier using the Miles and Misra method and the inoculated kits were incubated for 18-24 hours at 36-37° C. 10 μl was also used to inoculate blood agar in order to enumerate the viable count.

The results are shown in Table 2.

TABLE 2

| | | Number of colonies on |
|---|---|---|
| Organism | Colour change | blood agar |
| E. coli ESBL NCTC 13351 | Yes | 3 at 10⁻⁶ |
| E. coli ESBL NCTC 13352 | Yes | 4 at 10⁻⁶ |
| E. coli ESBL NCTC 13353 | Yes | 1 at 10⁻⁶ |

This shows that the detection kit can detect ESBLs at concentrations as low as 1-4 cfu/ml.

### Example 4

In order to test the shelf-life of the detection kit of the present invention, inoculations were carried out on a first set of kits which were stored at room temperature and a second set of kits which were refrigerated at 4° C., using an overnight broth of E. coli ESBL (NCTC 13351), E. coli (NCTC 10536) and Proteus mirabilis (NCTC 11938) which were tenfold serial diluted. Inoculations were carried out at day 0, week 1, week 2, week 3 and week 4 and colour changes were noted. A colour change from red (R) to yellow (Y) was taken as a positive result for ESBL.

The results are shown in Table 3

TABLE 3

| | Day 0 | Week 1 | | Week 2 | | Week 3 | | Week 4 | |
|---|---|---|---|---|---|---|---|---|---|
| | R.T. | R.T. | 4° C. | R.T. | 4° C. | R.T. | 4° C. | R.T. | 4° C. |
| ESBL 13351 10⁻⁵ | Y | Y | Y | Y | Y | Y | Y | Y | Y |
| ESBL 13351 10⁻⁶ | Y | Y | Y | Y | R | R | Y | Y | Y |
| ESBL 13351 10⁻⁷ | R | R | R | R | R | R | R | R | R |
| ESBL 13351 10⁻⁸ | R | R | R | R | R | R | R | R | R |
| E. coli neat | R | R | R | R | R | R | R | R | R |
| E. coli 10⁻¹ | R | R | R | R | R | R | R | R | R |
| E. coli 10⁻² | R | R | R | R | R | R | R | R | R |
| E. coli 10⁻³ | R | R | R | R | R | R | R | R | R |
| Proteus mirabilis neat | R | R | R | R | R | R | R | R | R |
| Proteus mirabilis 10⁻¹ | R | R | R | R | R | R | R | R | R |
| Proteus mirabilis 10⁻² | R | R | R | R | R | R | R | R | R |
| Proteus mirabilis 10⁻³ | R | R | R | R | R | R | R | R | R |

These results show that the kit is stable for at least 4 weeks at both room temperature and 4° C. It can be seen that no false positive results were obtained over the four week test period. The dilution of the ESBL detected remained constant over the test period. The slight variation in dilutions detected in weeks 2 and 3 is possibly due to sampling error. At such low dilutions, it is possible that the kits were inoculated with insufficient ESBL.

### Example 5

Detection kits according to the present invention were used in field trials using 23 patients in a ward known to have two ESBL positive patients. Swabs were taken of the groin area of all patients and each swab was dipped/swirled in the carrier of a respective detection kit at the patient's bedside. Three of the patients had catheters in situ and a 50 μl sample of urine was also added to the carrier of a detection kit.

The results are shown in Table 4.

TABLE 4

| | | Colour change | |
|---|---|---|---|
| | | using detection | |
| Patient | Specimen | kit | Culture results |
| 1 | swab | No | Neg |
| 1 | urine | No | Neg |
| 2 | swab | Yes | Pos |
| 3 | swab | Yes | Pos |
| 3 | urine | Yes | Pos |
| 4 | swab | No | Neg |
| 5 | swab | No | Neg |
| 6 | swab | Yes | Pos |
| 7 | urine | No | Neg |
| 8 | swab | No | Neg |
| 9 | swab | No | Neg |
| 10 | swab | No | Neg |
| 11 | swab | No | Neg |
| 12 | swab | No | Neg |
| 13 | swab | No | Neg |
| 14 | swab | No | Neg |
| 15 | swab | No | Neg |
| 16 | swab | Yes | Pos |
| 17 | swab | No | Neg |
| 18 | swab | No | Neg |
| 19 | swab | Yes | Pos |
| 20 | swab | No | Neg |
| 21 | swab | Yes | Pos |
| 22 | swab | Yes | Pos |
| 23 | swab | Yes | Pos |
| 246 | swab | No | Neg |

It can be seen that no false positive or false negative results were obtained using the detection kit of the present invention.

### REFERENCES

Carattoli A. Animal reservoirs for extended spectrum beta-lactamase producers. Clin Microbiol Infect. 2008 January; 14 Suppl 1:117-23.

Duan R S, Sit T H, Wong S S, Wong R C, Chow K H, Mak G C, Yam W C, Ng L T, Yuen K Y, Ho P L. (2006). Escherichia coli producing CTX-M beta-lactamases in food animals in Hong Kong. Microb Drug Resist 2006;12:145-148.

Mesa R J, Blanc V, Blanch A R, Cortes P, Gonzalez J J, Lavilla S, Miro E, Muniesa M, Saco M, Tortola M T, et al. Extended-spectrum beta-lactamase-producing Enterobacteriaceae in different environments (humans, food, animal farms and sewage). J Antimicrob Chemother 2006;58:211-215.

Meunier D, Jouy E, Lazizzera C, Kobisch M, Madec J Y. CTX-M-1- and CTX-M-15-type betalactamases in clinical Escherichia coli isolates recovered from food-producing animals in France. Int J Antimicrob Agents 2006;28:402-407.

Perez F, Endimiani A, Hujer K M, Bonomo R A. The continuing challenge of ESBLs. Curr Opin Pharmacol. 2007 October; 7(5):459-69. Epub 2007 Sep. 17. Review.

1. A detection kit for detecting the presence of ESBLs, the kit comprising a carrier containing:
a) at least one sugar;
b) at least one antibiotic for killing Gram positive bacteria;
c) at least one antifungal compound;
d) a mixture of cefpodoxime and at least one additional antibiotic for killing Gram negative bacteria, and/or a mixture of cefotaxime and ceftazidime; and
e) a pH indicator.

2. The detection kit according to claim 1 wherein the carrier is a liquid medium.

3. The detection kit according to claim 2 wherein the carrier is water and/or saline.

4. The detection kit according to claim 3 wherein the sugar, antibiotics/antifungal(s) and pH indicator are dissolved in the water/saline to form an aqueous solution.

5. The detection kit according to claim 2 wherein the carrier also include a pH buffer.

6. The detection kit according to claim 1 wherein the at least one sugar includes glucose and/or lactose.

7. The detection kit according to claim 1 wherein the at least one antibiotic for killing Gram positive bacteria is a glycopeptide antibiotic.

8. The detection kit according to claim 7 wherein the glycopeptide antibiotic is vancomycin or teicoplanin.

9. The detection kit according to claim 1 wherein the carrier contains a mixture of cefotaxime/ceftazidime and at least one additional antibiotic for killing Gram negative bacteria.

10. The detection kit according to claim 1 wherein the at least one additional antibiotic for killing Gram negative bacteria is a monobactam.

11. The detection kit according to claim 10 wherein the monobactam is aztreonam (AZT).

12. The detection kit according to claim 1 wherein the at least one antifungal compound is a polyene antimycotic and/or an imidazole/triazole antimycotic.

13. The detection kit according to claim 12 wherein the antifungal compound is amphotericin, nystatin or fluconazole.

14. The detection kit according to claim 1 wherein the pH indicator is phenol red.

15. The detection kit according to claim 1 wherein the carrier is contained within a transparent vessel.

16. A method for detecting the presence of ESBLs in a sample comprising:
a) inoculating the carrier of a detection kit as defined in claim 1 with the sample;
b) incubating the detection kit;
c) detecting a colour change in the pH indicator in the detection kit.

17. The method according to claim 16 where the sample is a clinical specimen.

18. The method according to claim 16 where the sample is an environmental sample.

19. The method according to claim 16 where the sample is a foodstuff.

20. The method according to claim 16 where the sample is a test sample from an animal.

21. The method according to claim 16 wherein the carrier of the detection kit is a liquid carrier and the inoculating is carried out by introducing the sample into the liquid carrier.

22. The method according to claim 21 wherein the inoculating is carried out by dipping/stirring a swab carrying the sample in the carrier.

23. The method according to claim 16 wherein the incubating is carried out for 18-24 hours at 36-37° C.

24. The method according to claim 16 wherein the detecting step is carried out by visually observing the colour of the pH indicator in the carrier.

25. The method according to claim 24 wherein the pH indicator is phenol red and the visual observance of a colour change from red to yellow indicates the presence of ESBL.

26. A detection kit substantially as any one embodiment herein described with reference to Example 1 or a method substantially as any one embodiment herein described with reference to Example 5.

27. (canceled)
